(19) United States
(12) Patent Application Publication
(10) Pub. No.: US 2025/0170306 A1
(43) Pub. Date: May 29, 2025

(54) IMPLANTABLE SYNTHETIC STRUCTURES COMPRISING POROUS POLYMER

(71) Applicant: CORNEAT VISION LTD, Ra'anana (IL)

(72) Inventors: Gilad LITVIN, MOSHAV SDE VARBURG (IL); Almog ALEY-RAZ, MOSHAV SDE VARBURG (IL)

(73) Assignee: CORNEAT VISION LTD, Ra'anana (IL)

(21) Appl. No.: 18/840,162

(22) PCT Filed: Feb. 23, 2023
(86) PCT No.: PCT/IL2023/050187

§ 371 Date: Aug. 21, 2024

Related U.S. Application Data
(60) Provisional application No. 63/313,285, filed on Feb. 24, 2022.

(51) Int. Cl. A61L 27/54; A61L 27/56; A61L 31/14; A61L 31/16

(52) U.S. Cl. CPC A61L 27/54; A61L 27/56; A61L 31/146; A61L 31/16; A61L 2430/02; A61L 2430/10; A61L 2430/24

(57) ABSTRACT

The invention provides synthetic structures comprising at least one porous polymer, being an implantable anatomical scaffold, such as orthopedic scaffold, processes for their preparation and uses thereof in implanting into the human body.

### BACKGROUND OF THE INVENTION

Bone replacement and other anatomical structure replacements have been known for the past century, for different clinical uses such as spine fusion, open-wedge tibial osteotomy, long bone fracture, oral and maxillofacial surgery, or periodontal treatments and so forth. There are many different bone substitute materials that have been used over the years, such as those derived from biological products, demineralized bone matrix, platelet-rich plasma, hydroxyapatite, adjunction of growth factors (like bone morphogenetic protein) or synthetic such as calcium sulfate, tri-calcium phosphate ceramics, bioactive glasses, or polymer-based substitutes. All these substitutes are not suitable for every clinical use, and they have to be chosen selectively depending on their purpose.

Damage or loss to the articular cartilage remains in the forefront of common problems encountered by orthopedic surgeons. The problem covers all ages, can cause significant morbidity, and can negatively affect function and quality of life. The treatment of chondral and osteochondral lesions has continued to be a major interest to orthopedic surgeons because most lesions do not heal spontaneously and may predispose the joint to subsequent development of secondary osteoarthritis.

Focal chondral defects are seen in up to 63% of patients undergoing arthroscopy of the knee, and it may even be more common in athletes. There are numerous surgical techniques aimed at cartilage restoration, including microfracture, osteochondral allograft, osteochondral autograft transfer system, and autologous chondrocyte implantation (ACI); however, these injuries present a complex problem. Microfracture has long been used as the primary technique to repair cartilage damage because of its ease of use and arthroscopic application. While this repair may afford the patient relief from pain and restoration of function for a limited time, the altered biomechanical and mechanical properties of fibrocartilage do not allow it to withstand the repetitive loads to the articular surface. Microfracture has been shown to have suboptimal results after short-term relief in athletic and elderly populations with failure of repair tissue leading to gradual deterioration of the joint surface and return of arthritic symptoms.

The main limitations to bone substitutes use remain the management of large defects and the lack of vascularization in their central part, which is likely to appear following their utilization.

There is a need to create an orthopedic anatomical scaffold, for example in cartilage restoration technique, that can restore normal function by regenerating hyaline cartilage in the defect while allowing integration of this repair cartilage to native tissue. There is a need to create a scaffold that is cost-effective, time efficient, single stage, and used without cell-based technology.

### SUMMARY OF THE INVENTION

The invention thus provides a synthetic structure comprising at least one porous polymer, being an implantable orthopedic scaffold.

The invention thus provides a synthetic structure comprising at least one porous polymer, being an implantable synthetic anatomical scaffold.

When referring to an implantable (anatomical) structure it should be understood to include a scaffold that is, at least in part, of a corresponding anatomical structure of a subject (for example at least a part of a limb, an organ, a bone structure, a muscle, cartilage, and so forth).

Said implantable structure is used for the purpose of being fixated to, interconnected to, bonded to, fused to, glued to, a part of the human body of a subject that is in need thereof, thus providing said subject with the ability to resume full functionality of the full anatomical structure that is implanted with said implant. In some embodiments, an implantable synthetic anatomical structure of the invention provides a reconstructed anatomical structure or at least a part thereof, capable of being implanted in a subject in need thereof.

In some embodiments, said implantable synthetic anatomical scaffold is an orthopedic scaffold. In some embodiments, said orthopedic scaffold is a scaffold that is, at least in part, of a corresponding an orthopedic anatomical structure of a subject.

In some embodiments, said an implantable structure is selected from a connecting bone to ligament, a muscle, tendon, a connecting bone to bone, a joint, a joint capsule, a synovial membrane, a cartilage and any combinations thereof.

In some embodiments, said structure further comprises additional connecting elements, such as for example a screw, a cable tie, a metal plate, a hook, a wire or any other high torque connection.

In some embodiments said orthopedic anatomical structure is an orthopedic anchor coated with a patch comprising at least one porous polymer.

In some other embodiments, said structure further comprises at least one soft tissue attachment side fashioned to connect with clamp, sutures, or other forms of connector to a polymeric, porous material intended for progressive, permanent integration.

In some embodiments said structure has a porous polymeric structure with pores of having pores of less than 5 microns. In some embodiments said structure has a porous polymeric structure with pores of between 5 to 20 microns.

In some embodiments said structure is biocompatible. In some embodiments said structure is biodegradable. In some embodiments said structure is non-biodegradable.

In some embodiments, said porous polymeric comprises nanofibers.

In some embodiments said porous polymeric comprises at least one porous electrospun polymer.

In some embodiments said structure further comprising a further synthetical artificial structure (additional bone replacement structure, metal implant, reinforcement structure, and so forth).

In some embodiments, said porous polymeric structure comprises at least one polymer selected from aromatic polyurethane, polycarbonate, poly(DTE carbonate) polycaprolactone (PCL), polylactic acid (PLA), poly-L-lactic acid (PLLA), Poly(DL-lactide-co-caprolactone, Poly(ethylene-co-vinyl acetate)vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof.

In some embodiments said structure further comprises at least one active agent.

In some embodiments, said at least one active agent is selected from a protein, collagen, fibronectin, or TGF-beta 2, heparin, growth factors, antibodies, antimetabolites, chemotherapeutic agents, anti-inflammatory agent, antibiotic agent, antimicrobial agent and any combinations thereof.

In some embodiments said implantable synthetic anatomical structure is selected from a bone structure, a cartilage structure, ligament, tendon and any combination thereof.

The invention further provides a synthetic structure as disclosed herein above and below for use in the treatment of an orthopedic condition or disorder.

In some embodiments, said orthopedic condition or disorder is selected from a ligamental rupture (partial or full thickness), anterior and/or posterior cruciate, Achilles rupture, cartilage damage (resulting in deficit especially in weight bearing joint), muscle tear or disinsertion, connecting muscle to muscle rupture, muscle to ligament rupture, interconnecting muscle/ligament complex to bone rupture and any combinations thereof.

The invention further provides a synthetic structure as disclosed herein above and below for use in ligament replacement and reconstruction.

The invention further provides a synthetic structure as disclosed herein above and below for use in orthopedic anchoring.

The invention further provides a synthetic structure as disclosed herein above and below for use in bone mounting.

The invention provides a synthetic structure as disclosed herein above and below for use in ligament replacement and reconstruction.

The invention provides a synthetic structure as disclosed herein above and below for use in the treatment of ligament disease, conditions or disorders.

The invention provides a synthetic structure as disclosed herein above and below for use in cartilage replacement and reconstruction. The invention provides a synthetic structure as disclosed herein above and below for use in the treatment of cartilage disease, conditions or disorders.

The invention provides a synthetic structure as disclosed herein above and below for use in tendon replacement and reconstruction.

The invention provides a synthetic structure as disclosed herein above and below for use in the treatment of tendon disease, conditions or disorders.

In some embodiments said implantable synthetic anatomical structure further comprises at least one sensor.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows an illustration of a ligament reconstruction using a structure of the invention. Black circles signify lock holes for externally attaching the artificial ligament to the bone-using Screw or Tack or any other fixation device.

FIGS. 2A-2E shows an illustration of cartilage repair structure of the present invention.

FIG. 3A shows an illustration of anatomical tendon reconstruction. FIG. 3B shows an embodiment of a structure of the invention.

FIGS. 4A-4B show a porous sleeve for orthopedic anchors—4A) Humerus Head being fixated with anchors. B) potential sleeve for orthopedic anchors. C) Orthopedic Anchor

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Ligament Replacement and Reconstruction

An elongated band of synthetic polymer intended for ligament reconstruction.

This band being made so that a zone of the band, the one coursing through bone tissue, is Porous, and the zone traversing the joint cavity is configured not to attach to surrounding structures (intra-articular) being a wire or a smooth material.

This band should have a sufficient tensile strength to withstand the wear and tear of said joint and can vary according to the specific joint. Anterior cruciate ligament substitute for example needs to withstand a load of over 2000 newton: “In the younger specimens, linear stiffness (242+/−28 N/mm) and ultimate load (2160+/−157 N) values found when the femur-ACL-tibia complex was tested in the anatomical orientation were higher than those reported previously (Woo S L, Hollis J M, Adams D J, Lyon R M, Takai S. Tensile properties of the human femur-anterior cruciate ligament-tibia complex. The effects of specimen age and orientation. Am J Sports Med. 1991 May-June; 19(3):217-25. doi: 10.1177/036354659101900303. PMID: 1867330).

Ligament replacement requires two safety mechanisms: Safety mechanism no.1—lock mechanism at the site of externalized artificial ligament. Safety mechanism no. 2—an intra-articular lock mechanism that connects the artificial ligament at its entrance to the joint cavity on both sides. This will give mechanical strength to the joint and spatial stability to the intra-osseous part until healing is complete and the porous material has integrated with the bone completely.

Both safety mechanisms are intended to: (i) relieve the pressure from the porous, intra-osseous, segments, (ii) to allow healing with some movement of the joint but no movement of the intra-osseous segments and to (iii) to prevent slippage of the artificial ligament either traumatic or gradual.

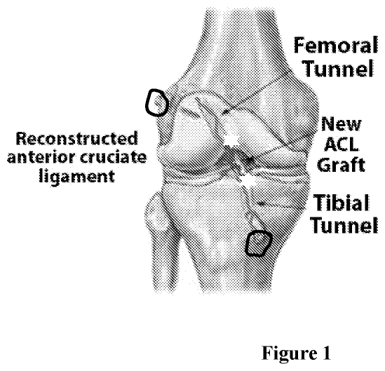

In FIG. 1 there is a schematic illustration of a ligament replacement of the invention. The purple signifies the smooth segment and the red the porous segment. Lock mechanism no. 2 location is shown by yellow stars. The artificial ligament is made of polymer. Central segment smooth, peripheral segments porous to allow tissue ingrowth. Strengthening element embedded into ligament structure-wire (nitinol or any other alloy). Said ligament further provides an elastic property.

### Cartilage Repair

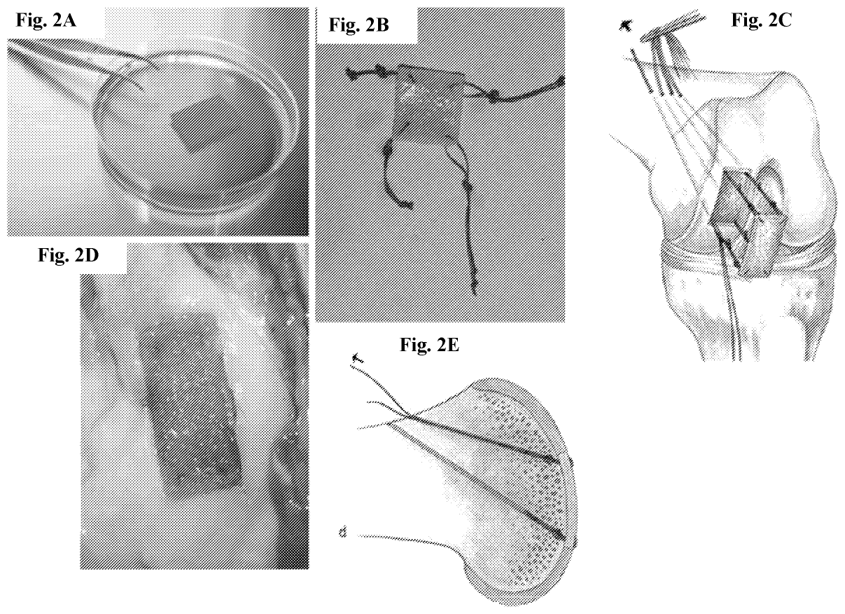

The majority of cartilage repair procedures are done in the knee or the ankle. Typically, cartilage does not heal at all and usually is replaced by scar tissue-called hyaline cartilage. The cartilage scaffold of the invention is a soft tissue substitute intended to replace damaged cartilage in any joint cavity. FIG. 2A-2E provide an illustration of embodiments of a cartilage repair scaffolds.

Scaffolds of the invention are designed to be chondroconductive and/or osteoconductive. Such scaffolds are implanted as a cell-free construct.

The porous material of the scaffold of the present invention spans the defect and promotes tissue growth through it, so it can withstand the shear forces and pounding of the bones.

In some embodiments, a scaffold of the invention is further impregnated with stem cells prior to implantation. In some other embodiments, a scaffold of the invention is capable of being implanted in deep gaps with optional attachment to the bone.

### Tendon Repair Embodiment

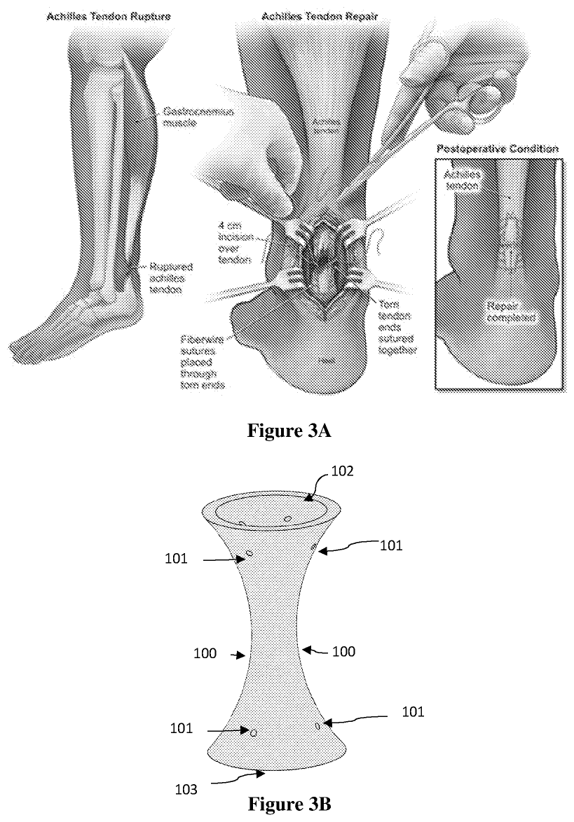

An example of the Achilles tendon is provided in FIG. 3.

Today simple suturing techniques are deployed when attempting to repair a torn tendon. In some embodiments of the scaffold of the invention, the structure is in the form of a sleeve with porous matrix facing inwards and smooth texture for improved sliding on the external face.

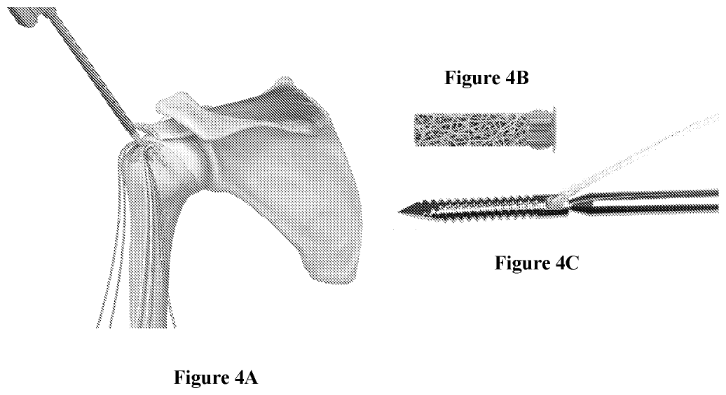

FIG. 3B shows a dual layer device, comprising a porous internal layer and a smooth external layer. Suturing holes (101) marked provide initial traction and immobility during the initial follow up period. A central narrowing (100) to add friction to the area that is being repaired. On both sides of the sleeve there should be widening to allow easy insertion of the muscular (102) and bony (103) attachment sites. FIGS. 4A-4B show a porous sleeve for orthopedic anchors. FIG. 4A shows the Humerus head being fixated with anchors. FIG. 4B shows a sleeve for orthopedic anchors of the invention. FIG. 4C shows an orthopedic anchor without the sleeve of the invention.

1. A synthetic structure comprising at least one porous polymer, being an implantable anatomical scaffold.

2. A synthetic structure according to claim 1, being an implantable orthopedic scaffold.

3. A synthetic structure according to claim 1, having a porous polymeric structure with pores of between pores of less than 5 microns.

4. A synthetic structure according to claim 1, having a porous polymeric structure with pores of between 5 to 20 microns.

5. A synthetic structure according to claim 1, wherein said porous polymeric structure comprises nanofibers.

6. A synthetic structure according to claim 1, wherein said porous polymeric structure comprises at least one porous electrospun polymer.

7. A synthetic structure according to claim 1, further comprising a further synthetic artificial structure.

8. A synthetic structure according to claim 1, wherein said porous polymeric structure comprises at least one polymer selected from aromatic polyurethane, polycarbonate, poly(DTE carbonate)polycaprolactone (PCL), polylactic acid (PLA), poly-L-lactic acid (PLLA), Poly(DL-lactide-co-caprolactone, Poly(ethylene-co-vinyl acetate)vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), and poly(L-lactide) or any combination thereof.

9. A synthetic structure according to claim 1, further comprising at least one active agent.

10. A synthetic structure, claim 1 further comprising at least one active agent selected from a protein, collagen, fibronectin, or TGF-beta 2, heparin, growth factors, antibodies, antimetabolites, chemotherapeutic agents, anti-inflammatory agent, antibiotic agent, antimicrobial agent and any combinations thereof.

11. A synthetic structure according to claim 1, wherein said scaffold is selected from a bone structure, cartilage, ligament, tendon, and any combination thereof.

12. A method of treating at least one orthopedic condition or disorder in a subject in need thereof, said method comprising implanting in said subject a synthetic structure comprising at least one porous polymer, being an implantable anatomical scaffold synthetic structure.

13. A method according to claim 12, wherein said orthopedic condition or disorder is selected from a ligamental rupture, anterior and/or posterior cruciate, Achilles rupture, cartilage damage, muscle tear or disinsertion, connecting muscle to muscle rupture, muscle to ligament rupture, interconnecting muscle/ligament complex to bone rupture and any combinations thereof.

14. (canceled)

15. (canceled)

16. (canceled)

17. (canceled)

18. (canceled)

19. An implantable synthetic anatomical structure according to claim 1, further comprising at least one sensor.
